(19) United States
(12) Patent Application Publication
(10) Pub. No.: US 2020/0171022 A1
(43) Pub. Date: Jun. 4, 2020

(54) FLT3 INHIBITORS FOR IMPROVING PAIN TREATMENTS BY OPIOIDS

(71) Applicants: INSERM (Institut National de la Santé et da la Recherche Médicale), Paris (FR); Université de Montpellier, Mantpellier (FR); Biodol Therapeutics, Clapiers (FR)

(72) Inventors: Jean VALMIER, Saint-Georges D'Orques (FR); Cyril RIVAT, Grabels (FR); Pierre SOKOLOFF, Ile aux Moines (FR)

(21) Appl. No.: 16/613,859

(22) PCT Filed: May 17, 2018
(86) PCT No.: PCT/EP2018/062945

§ 371 Date: Nov. 15, 2019

(30) Foreign Application Priority Data
May 17, 2017 (EP) 17305571.6

(51) Int. Cl. A61K 31/485; A61K 31/553; A61K 31/404; A61K 31/5377; A61K 31/517; A61K 31/44; A61K 31/4709; A61K 31/4453; A61K 39/395; A61K 45/06; A61P 23/00

(52) U.S. Cl. CPC A61K 31/485; A61K 31/553; A61K 31/404; A61K 31/5377; A61K 31/517; A61P 23/00; A61K 31/4709; A61K 31/4453; A61K 39/3955; A61K 45/06; A61K 31/44

(57) ABSTRACT

Inventors have evaluated the effects of the FLT3 inhibitors on morphine analgesic potency, on tolerance to morphine analgesia and on morphine-induced mechanical pain hypersensitivity. When the FLT3 inhibitor was administered together with morphine, the amount of analgesic effect was higher than that produced by morphine alone. Repeated administration of morphine induced a progressive decrease in morphine-induced analgesia as showed by the decreased percentage of MPE in control animals. Intrathecal pre-treatment with an FLT3 inhibitor reduced the decrease in morphine analgesia. The administration of FLT3 inhibitors completely prevented both the development of morphine-induced pain hypersensitivity and morphine-revealed latent pain sensitization. Accordingly, the invention relates to an FLT3 inhibitor for increasing the efficacy of an opioid for its analgesic effect, and hereby reducing the opioid dose while maintaining the opioid efficacy in a subject suffering from pain in need thereof.

### FIELD OF THE INVENTION

The present invention relates to the treatment of pain. More particularly, the invention relates to compounds and compositions used to improve the efficacy of opioids and prevent and treat their side-effects in a subject suffering from pain.

### BACKGROUND OF THE INVENTION

Although recent advances have been made in the therapeutic management of chronic pain, opioids remain the unsurpassed treatment for the management of acute and chronic pain. The prescribing of these medications has also become common over the past decade with more than 3% of adults in the United States receiving long-term opioid therapy for chronic non-cancer pain (Boudreau et al., 2009). Opioids are the most potent analgesic agents for the treatment of moderate to severe pain. Morphine, buprenorphine, fentanyl, oxycodone and methadone are the reference agents used in patients suffering from acute or chronic pain. In some conditions, in patients with terminal illnesses for instance, strong and repeated doses of opioid analgesics are needed. However, their use is seriously limited by undesirable side-effects such as constipation, nausea, vomiting, sedation and respiratory depression and there is a risk of abuse, addiction and number of opioid-associated deaths, as well (Kuehn, 2009). Most importantly, acute or chronic administration of an opioid can also produce tolerance to its analgesic effects, which requires increasing the dose of opioid and exacerbation of the aforementioned side-effects, and pain hypersensitivity referred as opioid-induced hyperalgesia (OIH) and latent pain sensitization (Rivat et al., 2002, 2007), which cannot be overcome by increasing the dose of the opioid and leaves the patient without adequate treatment of its pain and a considerable degradation of his quality of life.

Thus, there is a need to find new strategies to avoid or limit side-effects of opioids as described above and to retain the efficacy of opioids upon long-term treatment of chronic pain.

Document WO2011/083124 describes the use of a FLT3 receptor antagonist in the treatment or the prevention of neuropathic pain, said FLT3 receptor antagonist being a small organic molecule, an antibody and or an aptamer directed against FLT3 or FL, an inhibitor of the interaction between FL and FLT3 or an inhibitor of FLT3 expression selected from the group consisting of antisense RNA or DNA molecules, small inhibitory RNAs (siRNAs), short hairpin RNA and ribozymes.

Recently some FLT3 receptor antagonist were described in WO 2016/016370, which inhibit the interaction between FLT3 and FL.

### SUMMARY OF THE INVENTION

The invention relates to compounds to be used to reduce the dose of an opioid and thereby limiting its side effects, while preserving the said opioid efficacy to treat pain. The invention also relates to compounds used to prevent or treat tolerance or emergent pain hypersensitivity arising after treatment with an opioid. Those compounds are inhibitors of the Receptor Tyrosine Kinase FLT3 (for Fms-like tyrosine kinase 3) at therapeutically relevant doses.

### DETAILED DESCRIPTION OF THE INVENTION

### Definitions

effective amount: amount of a pharmaceutical compound which produces an effect on pain;
As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different. The 2 active ingredients be formulated as mixtures, only if they are administered simultaneously. They are formulated separately for the other administration schemes or regimens. For all the said types of administration, it may be repeated in particular during cycles or according to particular regimens.
As used herein, the term “the patient has not previously been treated by an opioid” means that, for the current pain that needs to be treated no opioid was administered to the patient. This does not mean that the patient did not ever receive an opioid treatment in the past.

Inventors have evaluated the effects of two methods for highly specific FLT3 inactivation, i.e. inhibition of Flt3 gene expression by Flt3-targeted small-interference RNA (siRNA) and Flt3 gene deletion and on tolerance to opioid analgesia and on opioid-induced mechanical pain hypersensitivity. Repeated administrations of opioids, such as morphine or buprenorphine, induced a progressive decrease in opioid-induced analgesia. Intrathecal pre-treatment with an Flt3-targeted siRNA prevented the development of tolerance to buprenorphine, whereas a scrambled siRNA had no effect (EXAMPLE 1). The development of tolerance is associated with the occurrence of long-term pain hypersensitivity and latent pain sensitization. The inventors have observed a significant decrease in the nociceptive threshold in rats treated with the opioid buprenorphine, i.e. mechanical pain hypersensitivity and the return to baseline values of the nociceptive threshold after cessation of treatment with buprenorphine. The administration of a single dose of buprenorphine precipitated pain hypersensitivity for 2 days, i.e. latent sensitization. The administration of an Flt3-targeted siRNA completely prevented both the development of buprenorphine-induced pain hypersensitivity and buprenorphine-revealed latent pain sensitization (EXAMPLE 2). Similarly, Flt3 gene deletion prevented the development of morphine-induced mechanical pain hypersensitivity (EXAMPLE 3). Altogether, these data demonstrate a potentiation of opioid analgesia by FLT3 inactivation. The present invention relates to compounds inactivating or inhibiting FLT3 to improve opioid analgesic effect for the management of acute and/or chronic pain.

Accordingly, in a first aspect, the invention relates to an FLT3 inhibitor for increasing the efficacy of an opioid for its analgesic effect, and hereby reducing the opioid dose while maintaining the opioid efficacy in a subject suffering from pain in need thereof.

Said increasing of the efficacy of the opioid and hereby reduction of the opioid dose is more particularly adapted for patients not having previously received opioid treatment.

Therefore, the present invention relates, according to a first embodiment, to a combination of an FLT3 inhibitor and an opioid, for use in the treatment of pain in a patient, wherein the patient has not previously been treated by an opioid, and in particular wherein the patient is firstly treated with an FLT3 inhibitor during a first phase of the treatment then treated with an opioid in a second phase of the treatment, and wherein said both phases may overlap.

According to a second embodiment, the invention relates to a combination of an FLT3 inhibitor and an opioid, for use in the treatment of pain in a patient previously treated by an opioid, for which an opioid-induced side-effect, has been stated.

According to third embodiment, the invention relates to a combination of an FLT3 inhibitor and an opioid, for use in the treatment of pain in a patient for which an ineffectiveness or a decline in a prior opioid treatment effectiveness or for which an opioid tolerance has been stated.

According to a fourth embodiment, the present invention relates to a pharmaceutical combination comprising an FLT3 inhibitor and an opioid.

According to a fifth embodiment, the present invention relates to a pharmaceutical combination comprising an FLT3 inhibitor, and in particular a compound of formula (I) or (II) as described herein after, and more particularly a FLT3 inhibitor compound as specifically listed herein after, and even more particularly N-(5-chloro-2-hydroxyphenyl)-3-(piperidin-1-ylsulfonyl)benzamide and an opioid for separate administration, administration spread out over time or simultaneous administration to a patient suffering from pain.

According to a sixth embodiment, the present invention relates to a pharmaceutical kit, in particular intended for treating pain, comprising:
(i) a first galenical formulation comprising an FLT3 inhibitor and in particular a compound of formula (I) or (II) as described herein after, and more particularly a FLT3 inhibitor compound as specifically listed herein after, and even more particularly N-(5-chloro-2-hydroxyphenyl)-3-(piperidin-1-ylsulfonyl)benzamide, and
(ii) a second galenical formulation comprising an opioid.

Compounds to improve the efficacy and safety of opioids As used herein, the terms “FLT3” or “FLT3 receptor” (Fms-related tyrosine kinase 3), also known as CD135, Ly72, Flk-2, Flt-3 or B230315G04, are used interchangeably and have their general meaning in the art. The FLT3 receptor can be from any animal species, but typically is a mammalian (e.g., human and non-human primate) FLT3 receptor, particularly a human FLT3 receptor. The naturally occurring human Flt3 gene has a nucleotide sequence as shown in Genbank Accession number NM_004119.2 and the naturally occurring human FLT3 protein has an aminoacid sequence as shown in Genbank Accession number NP_004110.2. The murine nucleotide and amino acid sequences have also been described (Genbank Accession numbers NM_010229.2 and NP_034359.2). The terms “FL” or “FLT3-Ligand” are used interchangeably and have their general meaning in the art. They refer to the cytokine which is a natural ligand of the FLT3 receptor. FL can be from any source, but typically is a mammalian (e.g., human and non-human primate) FL, particularly a human FL. The term “FLT3 inhibitor” refers to any compound which inhibits or down-regulates the biological activity associated with activation of the FLT3 receptor by FL in the subject, including any of the downstream biological effects otherwise resulting from the binding to FLT3 receptor with FL. Such a FLT3 inhibitor (e.g. a small organic molecule, an antibody directed against FLT3) can act by occupying the ligand binding site or a portion thereof of the FLT3 receptor, thereby making FLT3 receptor inaccessible to its natural ligand, FL, so that its normal biological activity is prevented or reduced. The term FLT3 receptor inhibitor includes also any agent able to interact with FL, the natural ligand of FLT3.

In a particular embodiment, the FLT3 inhibitor is a small organic molecule. The term “small organic molecule” as used herein, refers to a molecule of a size comparable to those organic molecules generally used in pharmaceuticals. The term excludes biological macro molecules (e. g. proteins, nucleic acids, etc.). Typically, small organic molecule weight ranges up to about 5000 Da, more preferably up to 2000 Da, and most preferably up to about 500 Da. Typically, FLT3 inhibitors are described in Sternberg et al. 2004 and in International Patent Application Nos WO 2002032861, WO 2002092599, WO 2003035009, WO 2003024931, WO 2003037347, WO 2003057690, WO 2003099771, WO 2004005281, WO 2004016597, WO 2004018419, WO 2004039782, WO 2004043389, WO 2004046120, WO 2004058749, WO 2004058749, WO 2003024969, WO 2006/138155, WO 2007/048088 and WO 2009/095399.

In a particular embodiment, the FLT3 inhibitor is an inhibitor of a receptor tyrosine kinase (RTKI) Examples of RTKI that are contemplated in the present invention include AG1295 and AG1296; Lestaurtinib (also known as CEP-701, formerly KT-5555, Kyowa Hakko, licensed to Cephalon); CEP-5214 and CEP-7055 (Cephalon); CHIR-258 (Chiron Corp.); GTP 14564 (Merk Biosciences UK). Midostaurin (also known as PKC 412 Novartis AG); MLN-608 (Millennium USA); MLN-518 (formerly CT53518, COR Therapeutics Inc., licensed to Millennium Pharmaceuticals Inc.); MLN-608 (Millennium Pharmaceuticals Inc.); sunitinib SU-11248 (Pfizer USA); SU-11657 (Pfizer USA); SU-5416 and SU-5614; THRX-165724 (Theravance Inc.); AMI-10706 (Theravance Inc.); VX-528 and VX-680 (Vertex Pharmaceuticals USA, licensed to Novartis (Switzerland), Merck & Co USA); and XL 999 (Exelixis USA). In a particular embodiment, the RTKI is selected from the group consisting of: lestaurtinib (CEP-701), sunitinib (SU-11248), midostaurin (PKC412), semaxinib (SU-5416), quizartinib (AC220), tandutinib (MLN518), sorafenib (BAY 43-9006), gilteritinib (ASP2215) and crenolanib (CP-868).

In a particular embodiment, the FLT3 inhibitor is a selective FLT3 receptor inhibitor. Examples of selective FLT3 receptor inhibitors that are contemplated by the invention include, but are not limited to, those described in (Hassanein et al., 2016) and in International Patent Applications No WO 2007/109120 and WO 2009/061446. In a particular embodiment, the FLT3 inhibitor is an FLT3 receptor small-molecule antagonist. Accordingly, in a particular embodiment, the selective FLT3 receptor antagonist is the compound known as AC220 or N-(5-tert-butyl-isoxazol-3-yl)-N′-{4-[7-(2-morpholin-4-yl-ethoxy)imidazo[2,1-b][1,3]benzothiazol-2-yl]phenyl}urea dihydrochloride, also known as quizartinib. Said AC220 compound may be made by methods known in the art, for example, as described in the international patent application WO 2007/109120. Another example of selective FLT3 inhibitor contemplated in the invention is gilteritinib, also known as ASP2215 (6-Ethyl-3-((3-methoxy-4-(4-(4-methylpiperazin-1-yl)piperidin-1-yl)phenyl)amino)-5-((tetrahydro-2H-pyran-4-yl)amino)pyrazine-2-carboxamide) described in WO2010128659.

In another embodiment, the FLT3 inhibitor is an inhibitor of the interaction between FLT3 and FL. The compounds that inhibit the interaction between FLT3 and FL encompass those compounds that bind either to the FLT3 receptor, FL or both, provided that the binding of the said compounds of interest prevents the interaction between FLT3 receptor and FL. Accordingly, said compounds may be selected from the group consisting of peptides, peptidomimetics, small organic molecules, antibodies, aptamers or nucleic acids. In a particular embodiment, the inhibitor of the interaction between FLT3 and FL is selected from one of the small organic molecules as described in the patent application W02016/016370. In a more particular embodiment, the inhibitor of the interaction between FL and FLT3 is N-(5-chloro-2-hydroxyphenyl)-3-(piperidin-1-ylsulfonyl)benzamide, also known as BDT001, which is described in Rivat et al. (2018).

As an inhibitor of the interaction between FLT3 and FL which is more particularly disclosed in WO2016/016370, the following compound of formula (I) (formula (2) in said document) may be cited, which can be implemented in the framework of the present invention:

a compound of general formula (I)

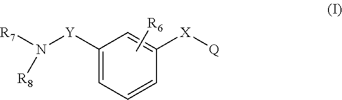

wherein:
X is CO—NH or triazolyl,
Y represents SO₂,
Q is selected from a group of formula:

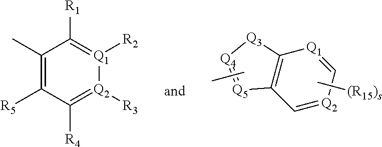

Q₁ and Q₂ are CH,
Q₃ is selected from O, S, N and NH,
Q₄ is selected from C and N, and CO,
Q₅ is selected from C and N,
R₆ is selected from H, OH, alkyl, hydroxyalkyl and alkoxy,
R₁ represents OH,
R₂ represents H,
R₃ is selected from H, OR₁₁, halo and O—(CH₂)ₚ—O-alkyl;
R₄ is selected from H, alkyl, halo, CN, trifluoromethyl, CO-alkyl, phenyl and benzyl; with the proviso that one from R₃ and R₄ is H;
R₅ is H, or
two from R₂ and R₃ or R₃ and R₄ or R₄ and R5 together with the carbon atoms to which they are attached form an aromatic ring comprising 5 to 6 members, and the others from R₂ to R₅ represent H,
R₇ and R₈ represent alkyl, or
R₇ and R₈ together with the N atom to which they are attached form a group of formulae:

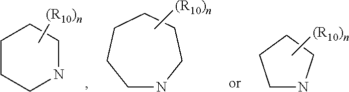

wherein R₁₀ is selected from H, alkyl, halo, trifluoromethyl, aryl and hydroxyalkyl or two adjacent R₁₀ groups together with the cyclic atoms to which they are attached form an aryl group; or
R₇ and R₈ together with the N atom to which they are attached form a group of formula:

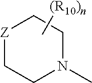

wherein Z is a NR₁₄ group, wherein R₁₄ is selected from phenyl, benzyl and pyrimidyl, or
R₇ is H and R₈ is cycloalkyl, preferably cyclohexyl and adamantyl,
R₁₁ is H or alkyl, R₁₅ represents a group selected from H, halo, OH and alkoxy,
s is 0, 1, 2 or 3, and
n is 1.

As an inhibitor of the interaction between FLT3 and FL which is still more particularly disclosed in WO2016/016370, the following compound of formula (II) (formula (2a) in said document) may be cited, which can be implemented in the framework of the present invention:

a compound of general formula (II)

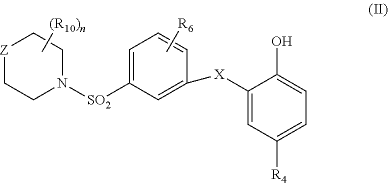

wherein
X is selected from a bond, CO, NH, CONH, NHCO and a 5- or 6-member heteroaromatic group comprising 2 or 3 N atoms;
Z is a bond or is selected from CHR₁₄, CH₂CHR₁₄, NR₁₄, CH₂NR₁₄ and O;
R₁₄ is selected from H, alkyl, cycloalkyl, aryl, and arylalkyl, wherein the cycloalkyl or aryl ring may comprise one or two heteroatoms in the cyclic structure selected from N and O and may be substituted with one or more substituent selected from alkyl, halo, cyano, amino, alkyl amino, dialkyamino, nitro, trifluoromethyl, aryl, alkyl-aryl, acyl, alkyloxy or aryloxy,
R₄ is selected from alkyl, halo, CN, trifluoromethyl, CO-alkyl, phenyl and benzyl,
R₆ is selected from H, OH, halo, alkyl, hydroxyalkyl and alkoxy,
R₁₀ is selected from H, alkyl, halo, trifluoromethyl, aryl and hydroxyalkyl or two adjacent R₁₀ groups together with the cyclic atoms to which they are attached form an aryl group; and
n is 0, 1 or 2.

Still specific compounds as disclosed in WO2016/016370, which are also suitable in the framework of the present invention are
N-(5-chloro-2-hydroxy-phenyl)-3-(1-piperidylsulfonyl)benzamide (BDT001);
N-(5-fluoro-2-hydroxy-phenyl)-3-(1-piperidylsulfonyl)benzamide;
N-(5-bromo-2-hydroxy-phenyl)-3-(1-piperidylsulfonyl)benzamide;
N-(2-hydroxy-5-phenyl-phenyl)-3-(1-piperidylsulfonyl)benzamide;
N-(5-benzyl-2-hydroxy-phenyl)-3-(1-piperidylsulfonyl)benzamide;
N-[2-hydroxy-5-(trifluoromethyl)phenyl]-3-(1-piperidylsulfonyl)benzamide;
N-(5-cyano-2-hydroxy-phenyl)-3-(1-piperidylsulfonyl)benzamide;
N-(5-acetyl-2-hydroxy-phenyl)-3-(1-piperidylsulfonyl)benzamide;
N-[5-(1,1-dimethylpropyl)-2-hydroxy-phenyl]-3-(1-piperidylsulfonyl)benzamide;
N-(2-hydroxy-4-methoxy-phenyl)-3-(1-piperidylsulfonyl)benzamide;
N-(3-hydroxy-2-naphthyl)-3-(1-piperidylsulfonyl)benzamide;
N-(2-hydroxy-1-naphthyl)-3-(1-piperidylsulfonyl)benzamide;
5-chloro-2-hydroxy-N-[3-(1-piperidylsulfonyl)phenyl]benzamide;
N-(5-chloro-2-hydroxy-phenyl)-4-methyl-3-(1-piperidylsulfonyl)benzamide;
N-(5-chloro-2-hydroxy-phenyl)-3-(dimethylsulfamoyl)benzamide;
N-(5-chloro-2-hydroxy-phenyl)-3-(cyclohexylsulfamoyl)benzamide;
3-(azepan-1-ylsulfonyl)-N-(5-chloro-2-hydroxy-phenyl)benzamide;
N-(5-chloro-2-hydroxy-phenyl)-3-[(2-methyl-1-piperidyl)sulfonyl]benzamide;
N-(5-chloro-2-hydroxy-phenyl)-3-[(3-methyl-1-piperidyl)sulfonyl]benzamide;
N-(5-chloro-2-hydroxy-phenyl)-3-[(4-methyl-1-piperidyl)sulfonyl]benzamide;
3-[(4-benzyl-1-piperidyl)sulfonyl]-N-(5-chloro-2-hydroxy-phenyl)benzamide;
N-(5-chloro-2-hydroxy-phenyl)-3-[[4-(1-piperidyl)-1-piperidyl]sulfonyl]benzamide;
N-(5-chloro-2-hydroxy-phenyl)-3-(4-methylpiperazin-1-yl)sulfonyl-benzamide;
N-(5-chloro-2-hydroxy-phenyl)-3-(4-phenylpiperazin-1-yl)sulfonyl-benzamide;
3-(4-benzylpiperazin-1-yl)sulfonyl-N-(5-chloro-2-hydroxy-phenyl)benzamide;
N-(5-chloro-1H-indol-7-yl)-3-(1-piperidylsulfonyl)benzamide;
5-chloro-3-[3-(1-piperidylsulfonyl)benzoyl]-1H-benzimidazol-2-one;
3-(1-adamantylsulfamoyl)-N-(5-chloro-2-hydroxy-phenyl)benzamide;
N-(5-chloro-2-hydroxy-phenyl)-3-[cyclohexyl(methyl)sulfamoyl]benzamide;
N-(5-chloro-2-hydroxy-phenyl)-3-[[2-(hydroxymethyl)-1-piperidyl]sulfonyl]benzamide;
N-(5-chloro-2-hydroxy-phenyl)-3-(4-pyrimidin-2-ylpiperazin-1-yl)sulfonyl-benzamide;
N-(5-chloro-2-hydroxy-phenyl)-3-[(3-phenyl-1-piperidyl)sulfonyl]benzamide;
N-(5-chloro-2-hydroxy-phenyl)-3-[[3-(hydroxymethyl)-1-piperidyl]sulfonyl]benzamide;
N-(5-chloro-2-hydroxy-phenyl)-3-pyrrolidin-1-ylsulfonyl-benzamide;
N-(5-chloro-2-hydroxy-phenyl)-3-morpholinosulfonyl-benzamide;
N-(5-chloro-2-hydroxy-phenyl)-3-indolin-1-ylsulfonyl-benzamide;
N-(2-chlorophenyl)-3-(1-piperidylsulfonyl)benzamide;
N-(2,5-dichlorophenyl)-3-(1-piperidylsulfonyl)benzamide;
N-(5-chloro-2-fluoro-phenyl)-3-(1-piperidylsulfonyl)benzamide;
N-(4-chloro-2-hydroxy-phenyl)-3-(1-piperidylsulfonyl)benzamide;
2-chloro-N-(5-chloro-2-hydroxy-phenyl)-5-(1-piperidylsulfonyl)benzamide;
N-(5-chloro-2-hydroxy-phenyl)-2-fluoro-5-(1-piperidylsulfonyl)benzamide;
N-(5-chloro-2-hydroxy-phenyl)-3-(cyclohexylsulfamoyl)-4-methyl-benzamide;
N-(5-chloro-2-hydroxy-phenyl)-3-(2-pyridylsulfamoyl)benzamide;
N-(5-chloro-2-hydroxy-phenyl)-2-methyl-5-(1-piperidylsulfonyl)benzamide;
N-(4-hydroxy-3-pyridyl)-3-(1-piperidylsulfonyl)benzamide;
2-hydroxy-N-(2-hydroxyphenyl)-5-(1-piperidylsulfonyl)benzamide;
N-(5-chloro-2-hydroxy-phenyl)-3-(2-phenylethylsulfamoyl)benzamide;
N-(5-chloro-2-hydroxy-phenyl)-3-(4-phenylbutylsulfamoyl)benzamide;
N-(5-chloro-2-hydroxy-phenyl)-3-(2-hydroxyethylsulfamoyl)benzamide;
N-[3-(1-piperidylsulfonyl)phenyl]-1H-indazol-3-amine;
4-chloro-2-[2-[3-(1-piperidylsulfonyl)phenyl]-1H-imidazol-5-yl]phenol;
3-[benzyl(cyclohexyl)sulfamoyl]-N-(5-chloro-2-hydroxy-phenyl)benzamide;
tert-butyl 2-[[3-[(5-chloro-2-hydroxy-phenyl)carbamoyl]phenyl]sulfonyl-cyclohexyl-amino]acetate;
N-(5-chloro-2-hydroxy-phenyl)-3-[cyclohexyl(3-phenylpropyl)sulfamoyl]benzamide;
N-(5-chloro-2-hydroxy-phenyl)-3-(4-hydroxybutylsulfamoyl)benzamide;
2-[[3-[(5-chloro-2-hydroxy-phenyl)carbamoyl]phenyl]sulfonyl-cyclohexyl-amino]acetic acid;
2-[3-(1-piperidylsulfonyl)phenyl]-3H-benzimidazol-4-ol;
3-[3-aminopropyl(cyclohexyl)sulfamoyl]-N-(5-chloro-2-hydroxy-phenyl)benzamide;
N-(3-aminopropyl)-3-(5-chloro-1,3-benzoxazol-2-yl)-N-cyclohexyl-benzenesulfonamide;
N-(5-chloro-2-hydroxy-phenyl)-3-[cyclohexyl(3-guanidinopropyl)sulfamoyl]benzamide;
N-(4,5-dichloro-2-hydroxy-phenyl)-3-(1-piperidylsulfonyl)benzamide;
5-chloro-N-[3-(1-piperidylsulfonyl)phenyl]-1H-indazol-3-amine;
N-(3-chloro-2-hydroxy-phenyl)-3-(1-piperidylsulfonyl)benzamide;
3-chloro-8-(1-piperidylsulfonyl)-5H-benzo [b][1,4]benzoxazepin-6-one;
3-chloro-8-(1-piperidylsulfonyl)-5,11-dihydrobenzo [b][1,4]benzodiazepin-6-one;
5-chloro-2-[3-(1-piperidylsulfonyl)phenyl]-1,3-benzoxazole;
4-chloro-2-[3-[3-(1-piperidylsulfonyl)phenyl]-1H-1,2,4-triazo1-5-yl]phenol;
7-chloro-2-[3-(1-piperidylsulfonyl)phenyl]-3H-benzimidazo1-4-ol;
5,7-dichloro-2-[3-(1-piperidylsulfonyl)phenyl]-3H-benzimidazol-4-ol;
4-[[3-[(5-chloro-2-hydroxy-phenyl)carbamoyl]phenyl]sulfonyl-cyclohexyl-amino]butanoic acid;
N-(5-chloro-2-hydroxy-phenyl)-3-[cyclohexyl(5-phenylpentyl)sulfamoyl]benzamide;
N-(5-chloro-2-hydroxy-phenyl)-3-[cyclohexyl(3-hydroxypropyl)sulfamoyl]benzamide;
N-(5-chloro-2-hydroxy-phenyl)-3-methyl-5-(1-piperidylsulfonyl)benzamide;
N-(5-chloro-2-hydroxy-phenyl)-3-[cyclopentyl(methyl)sulfamoyl]benzamide;
3-[2-aminoethyl(cyclohexyl)sulfamoyl]-N-(5-chloro-2-hydroxy-phenyl)benzamide;
N-(2-aminoethyl)-3-(5-chloro-1,3-benzoxazol-2-yl)-N-cyclohexyl-benzenesulfonamide;
3-[4-aminobutyl(cyclohexyl)sulfamoyl]-N-(5-chloro-2-hydroxy-phenyl)benzamide;
N-(4-aminobutyl)-3-(5-chloro-1,3-benzoxazol-2-yl)-N-cyclohexyl-benzenesulfonamide;
N-(5-chloro-2-hydroxy-phenyl)-3-[cycloheptyl(methyl)sulfamoyl]benzamide,
N-(3-aminopropyl)-3-[5-(5-chloro-2-hydroxy-phenyl)-1H-1,2,4-triazol-3-yl]-N-cyclohexyl-benzenesulfonamide;
N-(5-chloro-2-hydroxy-phenyl)-3-[cyclohexyl-[3-(dimethylamino)propyl]sulfamoyl]benzamide;
3-(5-chloro-1,3-benzoxazol-2-yl)-N-cyclohexyl-N-[3-(dimethylamino)propyl]benzenesulfonamide;
4-chloro-2-[4-[3-(1-piperidylsulfonyl)phenyl]triazol-1-yl]phenol;
4-chloro-2-[4-[3-(1-piperidylsulfonyl)phenyl]pyrimidin-2-yl]phenol;
N-(3-aminopropyl)-3-(1,3-benzothiazol-2-yl)-N-cyclohexyl-benzenesulfonamide;
3-[3-aminopropyl(cyclohexyl)sulfamoyl]-N-(2-methoxyphenyl)benzamide;
3-(1,3-benzoxazol-2-yl)-N-cyclohexyl-N-methyl-benzenesulfonamide;
3-[3-aminopropyl(cyclohexyl)sulfamoyl]-N-(2-hydroxyphenyl)benzamide;
N-(3-aminopropyl)-3-(1,3-benzoxazol-2-yl)-N-cyclohexyl-benzenesulfonamide;
3-[3-aminopropyl(cyclohexyl)sulfamoyl]-N-(2-hydroxy-3-methoxy-phenyl)benzamide;
N-(3-aminopropyl)-N-cyclohexyl-3-(7-methoxy-1,3-benzoxazol-2-yl)benzenesulfonamide;
N-(3-aminopropyl)-N-cyclohexyl-3-thiazolo[5,4-b]pyridin-2-yl-benzenesulfonamide;
2-[3-(1-piperidylsulfonyl)phenyl]benzotriazole;
N-(3-aminopropyl)-N-cyclohexyl-3-thiazolo[4,5-c]pyridin-2-yl-benzenesulfonamide;
N-(3-aminopropyl)-N-cyclohexyl-3-(7-hydroxy-1,3-benzoxazol-2-yl)benzenesulfonamide;
3-[3-aminopropyl(cyclohexyl)sulfamoyl]-N-(4,5-dichloro-2-hydroxy-phenyl)benzamide;
N-(3-aminopropyl)-N-cyclohexyl-3-(5,6-dichloro-1,3-benzoxazol-2-yl)benzenesulfonamide;
N-(3-aminopropyl)-3-(1H-benzimidazol-2-yl)-N-cyclohexyl-benzenesulfonamide;
N-(5-chloro-2-hydroxy-phenyl)-3-[cyclohexyl(3-morpholinopropyl)sulfamoyl]benzamide;
3-(5-chloro-1,3-benzoxazol-2-yl)-N-cyclohexyl-N-(3-morpholinopropyl)benzenesulfonamide;
3-(5-chloro-1,3-benzoxazol-2-yl)-N-cyclohexyl-N-(3-hydroxypropyl)benzenesulfonamide;
3-[5-(5-chloro-2-hydroxy-phenyl)-1H-1,2,4-triazol-3-yl]-N-cyclohexyl-N-methyl-benzenesulfonamide;
N-[5-chloro-2-hydroxy-4-(2-methoxyethoxy)phenyl]-3-(1-piperidylsulfonyl)benzamide;
N-(5-chloro-2-hydroxy-phenyl)-3-[cycloheptyl(methyl)sulfamoyl]benzamide;
ethyl 4-chloro-2-[[3-(1-piperidylsulfonyl)benzoyl]amino]benzoate;
N-(5-chloro-2-hydroxy-phenyl)-3-[(4-hydroxy-1-piperidyl)sulfonyl]benzamide;
4-chloro-2-[[3-(1-piperidylsulfonyl)benzoyl]amino]benzoic acid;
N-(3,5-dichloro-2-hydroxy-phenyl)-3-(1-piperidylsulfonyl)benzamide;
N-(5-chloro-1H-benzimidazol-2-yl)-3-(1-piperidylsulfonyl)benzamide;
N-(5-chloro-2-hydroxy-phenyl)-3-[(4,4-difluoro-1-piperidyl)sulfonyl]benzamide;
N-(3-acetyl-5-chloro-2-hydroxy-phenyl)-3-(1-piperidylsulfonyl)benzamide;
N-(5-chloro-2-hydroxy-3-methyl-phenyl)-3-(1-piperidylsulfonyl)benzamide;
N-[5-chloro-2-(1H-tetrazol-5-yl)phenyl]-3-(1-piperidylsulfonyl)benzamide;
N-[5-chloro-2-(N-hydroxycarbamimidoyl)phenyl]-3-(1-piperidylsulfonyl)benzamide;
N-(5-chloro-3-fluoro-2-hydroxy-phenyl)-3-(1-piperidylsulfonyl)benzamide; and
[4-chloro-2-[[3-(1-piperidylsulfonyl)benzoyl]amino]phenyl]dihydrogen phosphate.

In another particular embodiment, the FLT3 inhibitor is an anti-FLT3 antibody. The term “antibody” is thus used to refer to any antibody-like molecule that has an antigen binding region, and this term includes antibody fragments that comprise an antigen binding domain such as Fab', Fab, F(ab')2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art. In some embodiments, the antibody is a monoclonal antibody. In some embodiments, the antibody is non-internalizing. As used herein the term “non-internalizing antibody” refers to an antibody, respectively, that has the property of to bind to a target antigen present on a cell surface, and that, when bound to its target antigen, does not enter the cell and become degraded in the lysosome. Particularly, in the context of the invention, the antibody is a single domain antibody. The term “single domain antibody” has its general meaning in the art and refers to the single heavy chain variable domain of antibodies, which are naturally devoid of light chains; such antibodies can be found in Camelid mammals. Such single domain antibody are also called VHH or “nanobody®”. For a general description of (single) domain antibodies, reference is also made to the prior art cited above, as well as to EP 0 368 684, (Holt et al., 2003; Ward et al., 1989); and WO 06/030220, WO 06/003388. In the context of the invention, the amino acid residues of the single domain antibody are numbered according to the general numbering for VH domains given by the International ImMunoGeneTics information system aminoacid numbering (http://imgt.cines.fr/). Particularly, in the context of the invention, the antibody is a single chain variable fragment. The term “single chain variable fragment” or “scFv fragment” refers to a single folded polypeptide comprising the VH and VL domains of an antibody linked through a linker molecule. In such a scFv fragment, the VH and VL domains can be either in the VH-linker-VL or VL-linker-VH order. In addition to facilitate its production, a scFv fragment may contain a tag molecule linked to the scFv via a spacer. A scFv fragment thus comprises the VH and VL domains implicated into antigen recognizing but not the immunogenic constant domains of corresponding antibody. In a particular embodiment, the anti-FLT3 antibody is an anti-FLT3 neutralizing antibody such as IMC-EB10 (also known as LY3012218) and IMC-NC7 described in (Li et al., 2004) and in US patent application No US 2009/0297529. In a particular embodiment, the FLT3 inhibitor is an anti-FL antibody. In another embodiment the FLT3 inhibitor is an aptamer directed against FLT3 or FL. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library.

In another particular embodiment, the FLT3 inhibitor is an inhibitor of Flt3 gene expression. In some embodiments, the inhibitor of Flt3 expression is an antisense oligonucleotide. Anti-sense oligonucleotides, including anti-sense RNA molecules and anti-sense DNA molecules, would act to directly block the translation of Flt3 mRNA by binding thereto and thus preventing protein translation or increasing mRNA degradation, thus decreasing the level of FLT3 proteins, and thus activity, in a cell. For example, antisense oligonucleotides of at least about 15 bases and complementary to unique regions of the mRNA transcript sequence encoding Flt3 can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In some embodiments, the inhibitor of Flt3 expression is a small interference RNAs (siRNAs). Flt3 gene expression can be reduced by contacting the subject or cell with a small double stranded RNA (dsRNA), or a vector or construct causing the production of a small double stranded RNA, such that FLT3 expression is specifically inhibited (i.e. RNA interference or RNAi). Methods for selecting an appropriate dsRNA or dsRNA-encoding vector are well known in the art for genes whose sequence is known (e.g. see (McManus and Sharp, 2002; Tuschl et al., 1999); U.S. Pat. Nos. 6,573,099 and 6,506,559; and International Patent Publication Nos. WO 01/36646, WO 99/32619, and WO 01/68836).

In some embodiments, the inhibitor of Flt3 expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of FLT3 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of FLT3 expression is an endonuclease. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the error prone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in (Zetsche et al., 2015).

In a particular embodiment, the inhibitor of Flt3 gene expression as described above may be delivered in vivo alone or in association with a vector. In its broadest meaning, a “vector” is any vehicle capable of facilitating the transfer of the antisense oligonucleotide of the invention to the cells. Preferably, the vector transports the nucleic acid to cells with reduced degradation relative to the extent of degradation that would result in the absence of the vector. In general, the vectors useful in the invention include, but are not limited to, naked plasmids, non-viral delivery systems (electroporation, sonoporation, cationic transfection agents, liposomes, etc . . . ), phagemids, viruses, other vehicles derived from viral or bacterial sources that have been manipulated by the insertion or incorporation of the antisense oligonucleotide nucleic acid sequences. Viral vectors are a preferred type of vector and include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus. One can readily use other vectors not named but known to the art. Typically, viral vectors according to the invention include adenoviruses and adeno-associated (AAV) viruses, which are DNA viruses that have already been approved for human use in gene therapy. Currently, 12 different AAV serotypes (AAV1 to 12) are known, each with different tissue tropisms (Wu et al., 2006). Recombinant AAV are derived from the dependent parvovirus AAV (Choi et al., 2005). The adeno-associated virus type 1 to 12 can be engineered to be replication deficient and is capable of infecting a wide range of cell types and species (Wu et al., 2006). It further has advantages such as, heat and lipid solvent stability; high transduction frequencies in cells of diverse lineages, including hematopoietic cells; and lack of superinfection inhibition thus allowing multiple series of transductions. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by, intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

As used herein, the terms “opiate” and “opioid” are used interchangeably and mean any natural or chemical compound with morphine-like pharmacological activities that are mediated by the activation of opioid receptors. Opioid receptors are members of the G protein coupled receptor (GPCR) superfamily characterized by the presence of seven transmembrane regions. Three distinct type of receptors, namely mu (MOP), delta (DOP) and kappa (KOP) have been identified. Opioid receptors belong to the well-known Gi/o class of GPCRs. It is commonly accepted that the main inhibitory effects of opioid on pain transmission are due to the stimulation of μ-opioid receptor (MOP) resulting in an inhibition of adenylyl cyclase and ion channels. In some embodiments, the opioid refers to natural and synthetic opiates which are known or which will be developed in the future. In some embodiments, the opioid is selected from the group consisting of: alfentanil, allylprodine, alphaprodine, anileridine, benzylmorphine, bezitramide, buprenorphine, butorphanol, clonitazene, codeine, cyclazocine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetylbuturate, dipipanone, eptazocine, ethoheptazine, ethylmethylthiambutene. ethylmorphine, etonitazene fentanyl, heroin, hydrocodone, hydromorphone, hydroxypethideine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentonil, tilidine or tramadol. In a particular embodiment, the opiate is morphine. In a particular embodiment, the opioid is buprenorphine.

As used herein, the term “efficacy” refers to receptor signaling efficacy, or the magnitude of a receptor-mediated effect produced by a drug relative to receptor occupancy. The efficacy of an opioid can be measured by the known methods in the art. For example, such methods are described in Michael et al 2011, British Journal of Pharmacology (2011).

As used herein, the term “analgesic effect” refers to a clinical effect which results from the use of a substance that produces analgesia. The term “analgesia” refers to loss of sensitivity to pain without loss of consciousness. Typically, an opioid is used to reduce the sensitivity to pain, such opioid is called an analgesic drug. In a particular embodiment, the analgesic drug is morphine. In another particular embodiment, the analgesic drug is buprenorphine.

In a particular embodiment, the FLT3 inhibitor as described above is suitable for reducing an opioid-induced side-effect, such as abuse or addiction, nausea, constipation or respiratory depression. The inventors have found that administering an FLT3 receptor inhibitor concomitantly with morphine produces a greater analgesic effect than that produced by morphine alone and that the combination of sunitinib and morphine permits a reduction by 50% of the dose of morphine, i.e. morphine dose-sparing, while maintaining the efficacy of the opioid (EXAMPLE 4).

In another embodiment, an inhibitor of the interaction between FLT3 and FL can be used in combination with an opioid to produce a greater analgesic effect than that produced by morphine alone (EXAMPLE 4). Remarkably, BDT001, an inhibitor of the interaction between FLT3 and FL, has no analgesic effect per se, but potentiates the analgesic effect of morphine, which permits a reduction of 30% of the dose of morphine to obtain the same analgesic effect (EXAMPLE 4).

Because the side-effects of morphine, such as constipation, nausea, vomiting, sedation and respiratory depression are dose-dependent, the reduction of the dose of opioid will reduce the intensity of these side-effects.

Therefore, the present invention further relates to a combination of an FLT3 inhibitor and an opioid, for use in the treatment of pain in a patient, wherein the patient has not previously been treated by an opioid and the opioid is administered in a daily dose reduced by at least 20%, at least 30%, or even at least 40% in comparison to a dose adapted to the treatment of the same pain for the same patient, in absence of FLT3 inhibitor.

Moreover, other risks associated with morphine, such as abuse and addiction, or appearance of pain hypersensitivity (IOH) and latent pain sensitization, will also be reduced.

The inventors have evaluated the effects of the FLT3 receptor inhibitor sunitinib on tolerance to morphine analgesia. They have shown that a repeated administration of morphine twice a day for 4 days induced a progressive decrease in morphine-induced analgesia as showed by the decreased percentage of MPE in control animals (EXAMPLE 5). Similar results were obtained with another opioid, buprenorphine and another FLT3 receptor inhibitor, lestaurtinib, also known as CEP-701 (EXAMPLE 5). Also, similar results were obtained with BDT001, an inhibitor of the interaction between FLT3 and FL. (EXAMPLE 5). The results show that FLT3 inhibitors or an inhibitor of FLT3 expression are able to prevent part of the tolerance to opioid analgesia. Accordingly, in a particular embodiment, the FLT3 receptor inhibitor or an inhibitor of FLT3 expression is used to prevent opioid tolerance, i.e. the loss of opioid efficacy on pain upon repeated treatment with the said opioid.

The inventors have shown that when the FLT3 inhibitor (sunitinib), or BDT001 was administered, morphine-induced pain hypersensitivity was completely prevented (EXAMPLE 6).

Accordingly, in a particular embodiment, the FLT3 receptor inhibitor or an inhibitor of FLT3 expression is used to prevent opioid tolerance or opioid-induced pain sensitization and latent pain sensitization. Accordingly, the FLT3 inhibitor according to the invention, or an inhibitor of FLT3 expression, is combined with an opioid as described above for use in a method for preventing or treating a subject suffering from at least one of the side-effects on pain induced by opioids. As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “side effects” has its general meaning in the art and refers to unintended effect occurring at normal dose related to the pharmacological properties. In the context of the invention, the term “side effects on pain” the side effects are induced by the administration of opioids such as morphine in a subject suffering from a pain. In some embodiments, the side effects on pain induced are selected from the group consisting of: opioid tolerance (OT); opioid-induced hyperalgesia (OIH); opioid sensitization. In a particular embodiment, the side effect is opioid tolerance (OT). As used herein, the term “opioid tolerance” (OT) refers to a progressive loss of response to an opioid that can be overcome by increasing the dose. It is mainly due to changes in numbers of receptors, signalling proteins and levels of opioid receptor phosphorylation are part of the alterations that reflect cellular adaptive changes to opioid exposure. In a particular embodiment, the side effect is opioid-induced hyperalgesia (OIH). As used herein, the term “opioid-induced hyperalgesia” (OIH) refers to a sensitization process by which opioids, paradoxically, cause pain hypersensitivity (an inflammation or tissue or nerve damage). In a particular embodiment, the side effect is opioid-induced latent pain sensitization. As used herein, the term “opioid-induced latent pain sensitization” refers to the persistence of central sensitization in the absence of behavioural signs of hypersensitivity. Latent pain sensitization is a form of long-lasting pain vulnerability that develops after traumatic injury stress or opioid administration, by which the organism may demonstrate greater susceptibility to a potentiated pain response upon subsequent injury or stressor or opioid exposure.

As used herein, the term “subject” denotes a mammal, such as a rodent, a feline, a canine, and a primate. Particularly, the subject according to the invention is a human. More particularly, the subject according to the invention has or susceptible to have acute or chronic pain.

In a particular embodiment, the side effect is opioid tolerance (OT). In another embodiment the side effect is opioid-induced hyperalgesia (OIH). In a further embodiment, the side effect is opioid-induced latent pain sensitization.

More particularly, the FLT3 inhibitor according to the invention is administered simultaneously, separately or sequentially to a subject suffering from at least one of the side-effects with an opioid as described above.

Doses and Regimen

The treatment is continuous for only from FLT3 inhibitor and opioid or for both or non continuous for only from FLT3 inhibitor and opioid or for both.

A “continuous treatment” means a long-term treatment, which can be implemented, including with various administration frequencies, preferably twice a day, and more preferably once a day.

Administration of the FLT3 inhibitor and the opioid may be simultaneous, separate or spread out over time.

The combination can be administered repeatedly over the course of several sequences or cycles according to a protocol which depends on the nature of the pain and intensity of the pain to be treated and also on the patient to be treated (age, weight, previous treatment(s), etc.). The protocol can be determined by any practitioner specializing in pain.

Various sequences or cycles of administration respectively of the FLT3 inhibitor and the opioid may be implemented within the framework of the present invention.

According to a preferred embodiment, FLT3 inhibitor is administered before the opioid. Its administration may then of course be repeated along a long-term treatment, preferably provided that the opioid is administered only once a minimal dose of FLT3 inhibitor is present in the blood of the patient. The dose of the FLT3 inhibitor should be adapted according to the characteristics of the individual subject to be treated (age, weight), so that the maximal blood level of the FLT3 inhibitor ranges from 10 to 1000 ng/mL, preferably from 20 to 200 ng/mL.

According to a preferred embodiment, and to one of the possible sequences of administration, the FLT3 inhibitor is administered to the patient during a first phase of treatment and the patient is then treated with an opioid in a second phase of the treatment. Said both phases may overlap or not.

According to some embodiments, the invention further relates to a method for treating pain in a patient in need thereof, consisting of:
(i) administering to a patient in need thereof an effective amount of FLT3 inhibitor during a first phase of treatment; and
(ii) then administering to said patient in need thereof an effective amount of an opioid in a second phase of treatment, wherein the first and second phase of treatment may overlap or not and wherein the sequence (i) and (ii) may be repeated, in particular so as to maintain a minimal level of the FLT3 inhibitor in the blood of the patient, which may range from 1 to 100 ng/mL, in particular from 2 to 50 ng/mL and preferably from 5 to 20 ng/mL.

According to a particular embodiment, the present invention relates to a combination of an FLT3 inhibitor and an opioid, for use in the treatment of pain in a patient, according to anyone of the three first embodiments as described above, wherein a minimal level of FLT3 inhibitor is maintained in the blood of the patient ranging from 1 to 100 ng/mL, in particular from 2 to 50 ng/mL and preferably from 5 to 20 ng/mL.

In the herein above described embodiments, the first phase of treatment may for example last 10 minutes to 30 days, for example 1 hour to 10 days, and more particularly 30 minutes to 1 day. The second phase of treatment may for example last 1 day to 6 months, for example 10 days to 2 months, and more particularly 4 days to 10 days.

The overlap between the two phases may last 1 day to 6 months, for example 10 days to 2 months, and more particularly 1 day to 4 days.

According to a particular embodiment, the two phases do not overlap, and the two phases are separated by a period of time or interval lasting between 30 minutes and 10 days in particular 1 hour and 4 days.

The person responsible for administration will, in any event, determine the appropriate dose for the individual subject.

Typically, the daily dose of FLT3 inhibitor during the first phase of administration may range between 0.1 and 1000 mg, in particular between 1 and 100 mg, more particularly between 5 and 20 mg.

The dosage of the opioid may then be reduced by at least 20%, 30% or even 50% in comparison to the dosage useful for the treatment of the same pain for the same patient without a previous administration of FLT3 inhibitor. Typically, opioid may then be administered in a daily dose ranging from 0.01 to 1000 mg, in particular from 0.1 to 100 mg and more particularly from 1 to 20 mg.

As an alternative of the regimen as described above, the administration of FLT3 inhibitor may be not continuous whereas the administration of the opioid is continuous or not.

As another alternative of the sequential administration as described above, the FLT3 inhibitor and the opioid may be administered in a unique dosage form or unit pharmaceutical preparation.

In a particular embodiment the pharmaceutical combination according to the invention, comprising an amount of an FLT3 inhibitor ranging from 0.5 to 500 mg, in particular from 2 and 100 mg more particularly between 5 and 20 mg and an amount of an opioid ranging from 0.01 to 1000 mg, more particularly from 0.1 to 100 mg, and even more particularly from 1 to 20 mg.

All combinations of doses, frequencies and treatment period are encompassed within the scope of the present invention.

In a more particular embodiment, the FLT3 inhibitor is administered before predictable pain occurs. For example, it is well known that moderately or highly invasive surgical procedures, such as cardiac operations, joint replacement, tumour extraction, digestive tract partial ablation, graft or amputation elicit, during the hours and days following the procedure or even longer, pain of various intensity, which requires the use of opioids. In these conditions, and according to a particular embodiment, the FLT3 inhibitor may be administered during a surgical procedure, when the subject is anesthetized and before initiation of the opioid treatment, which generally takes place during the post-operative care.

Pharmaceutical Compositions

The invention further relates to a pharmaceutical combination comprising an FLT3 inhibitor, and in particular a compound of formula (I) or (II) as described above, and more particularly a FLT3 inhibitor compound as specifically listed herein above and even more particularly N-(5-chloro-2-hydroxyphenyl)-3-(piperidin-1-ylsulfonyl)benzamide also known as BDT001, and an opioid for separate administration, administration spread out over time or simultaneous administration to a patient suffering from pain.

The invention further relates to a pharmaceutical kit, in particular intended for treating pain, comprising:
(i) a first galenical formulation comprising an FLT3 inhibitor, and
(ii) a second galenical formulation comprising an opioid.

The FLT3 inhibitors as described above may be combined with pharmaceutically acceptable excipients, and optionally sustained-release matrices, such as biodegradable polymers, to form pharmaceutical compositions. In a particular embodiment, the invention relates to a pharmaceutical combination comprising an FLT3 inhibitor and an opioid, as a pharmaceutical composition.

In a particular embodiment, the pharmaceutical combination according to the invention, comprises an FLT3 inhibitor, which is selected from the group consisting of lestaurtinib (CEP-701), sunitinib (SU-11248), midostaurin (PKC412), semaxinib (SU-5416), quizartinib (AC220), tandutinib (MLN518), sorafenib (BAY 43-9006), gilteritinib and crenolanib (CP-868).

In another particular embodiment, the pharmaceutical combination according to the invention, comprises an inhibitor of the interaction between FL and FLT3; In a more particular embodiment, the inhibitor of the interaction between FLT3 and FL is selected from those described in WO2016/016370 or one of the compound of formula (I) or (II) as described above, and more particularly a FLT3 inhibitor compound as specifically listed herein above. In an even more particular embodiment, the pharmaceutical combination according to the invention, comprises BDT001.

In a particular embodiment, the pharmaceutical combination according to the invention, comprising the opioid which is selected from the group consisting of fentanyl, alfentanil, codeine, pethidine, remifentanyl, morphine, tramadol, buprenorphine, nalbuphine, morphine sulphate, hydromorphone hydrochloride, coated morphine sulphate. In a particular embodiment, the pharmaceutical composition according to the invention, wherein the dose of the opioid is reduced compared to when it is administered alone.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The peptide or the drug conjugate (or the vector comprising peptide or the drug conjugate) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1,000 ml of hypodermoclysis fluid or injected at the proposed site of infusion. Some variation in dosage will necessarily occur depending on the condition of the subject being treated.

The invention will be further illustrated by the following FIGURES and EXAMPLES. However, these figures and examples should not be interpreted in any way as limiting the scope of the present invention.

### FIGURES

FIG. 1: Inhibition of FLT3 gene expression by an Flt3-targeted siRNA abolishes tolerance to buprenorphine in rats. Buprenorphine (100 μg/kg s.c.) was administered twice a day (morning and evening) for 4 consecutive days. Intrathecal injection of saline or an siRNA directed against FLT3 (2 μg/rat) or a scrambled siRNA was performed once daily (morning), 1 h before each opioid injection. Nociceptive threshold was measured 2 h before and 30 min after opioid administration performed on the morning and once daily. Results are expressed as percentage of maximal potential analgesic effect (MPE)±S.E.M. *P<0.05 compared with animals treated by the opioid alone.

FIG. 2: Effects of the inhibitor of Flt3 gene expression (B) on buprenorphine-induced hyperalgesia and latent pain sensitization. Buprenorphine (100 μg/kg s.c.) or saline was administered twice a day (morning and evening) for 4 consecutive days. Intrathecal injection of saline, an siRNA directed against FLT3 (2 μg/rat) or a scrambled siRNA (2 μg/rat) was performed once daily (morning), 1 h before each opioid injection. Nociceptive threshold was measured 2 h before and 30 minutes after opioid administration performed on the morning and once daily. On D12, a single buprenorphine injection (B, 100 μg/kg, s.c.) was performed in each group and nociceptive threshold was evaluated 30 minutes later. *P<0.05 compared with animals treated with buprenorphine alone.

FIG. 3: Flt3 gene deletion in the mouse abolishes mechanical hypersensitivity appearing after repeated administrations of morphine. Morphine (10 mg/kg i.p.) or saline was administered twice a day (morning and evening) for 4 consecutive days. Nociceptive threshold was measured by the von Fey test before opioid administration on D2 and on 12 h after the last morphine injection D5. In wild-type (F1t3+/+) animals, repeated morphine administrations produced a decrease in nociceptive threshold, i.e. mechanical hypersensitivity, which was completely prevented Flt3-deficient (F1t3−/−) animals. Results are Mean±S.E.M. *** P<0.001 compared to saline WT.

FIG. 4: Dose-sparing effects of FLT3 inhibitor sunitinib on morphine-induced analgesia.

Morphine was administered at a dose of either 0.5, 1, 1.5 or 2 mg/kg, either alone or in combination with sunitinib administered at the dose of 3 mg/kg, 90 min before morphine, and the paw pressure vocalization threshold was measured at 30 min, 60 min, 90 min, 120 min and 150 min after morphine administration. Then, the amount of analgesia was calculated as the Area Under the Curve of the analgesic effect as a function of time. Results are Mean±S.E.M. *P<0.05 compared to morphine alone.

FIG. 5: Dose-sparing effects of BDT001, an inhibitor of the interaction between FLT3 and FL on morphine-induced analgesia.

Morphine was administered at a dose of either 0.5, 1, 1.5 or 2 mg/kg, either alone or in combination with sunitinib administered at the dose of 3 mg/kg, 90 min before morphine, and the paw pressure vocalization threshold was measured at 30 min, 60 min, 90 min, 120 min and 150 min after morphine administration. Then, the amount of analgesia was calculated as the Area Under the Curve of the analgesic effect as a function of time. Results are Mean±S.E.M. *P<0.05 compared to morphine alone.

FIG. 6: Effects of the FLT3 inhibitor sunitinib on tolerance to morphine-induced analgesia. Morphine (2 mg/kg s.c.), or buprenorphine (100 μg/kg s.c.) was administered twice a day (morning and evening) for 4 consecutive days. Intrathecal injection of saline, sunitinib (53 ng/rat), was performed 1 h before each morphine injection. Nociceptive threshold was measured 2 h before and 30 min after opioid administration performed on the morning and once daily. Results are expressed as percentage of maximal potential effect (MPE)±S.E.M. *P<0.05 compared with animals treated by morphine alone.

FIG. 7: Effects of the FLT3 inhibitor sunitinib on morphine-induced hyperalgesia and latent pain sensitization. Morphine (2 mg/kg s.c.) or saline was administered twice a day (morning and evening) for 4 consecutive days. Intrathecal injection of saline, sunitinib (53 ng/rat) was performed once daily (morning), 1 h before each morphine injection. Nociceptive threshold was measured 2 h before and 30 min after morphine administration performed on the morning and once daily. On D12, a single morphine (2 mg/kg; s.c.) was performed in each group and nociceptive threshold was evaluated 30 min later. *P<0.05 compared with animals treated by morphine alone.

### EXAMPLES

### Example 1: Reduction by an Inhibitor of FLT3 Expression of Tolerance After Chronic Buprenorphine Treatment

Materials & Methods

Animals

Male Sprague-Dawley rats (Janvier, Le Genest St Isle, France) weighing 175-199 g were used for the different experiments described here. Rats were kept under controlled environmental conditions (22° C., 60% relative humidity, 12 h light/dark cycle with lights on at 7:00 A.M., food and water ad libitum). To limit the stress induced by the experimental procedure, the general plan for an experimental phase was as follows:
arrival and housing of the animals: 4 days;
acclimatization of the animals for 10 days to the experimental conditions in order to avoid any possibility of measurement bias being induced by stress;
evaluation of nociceptive threshold baseline
repeated opioid administration and evaluation of treatment effects

Nociceptive Testing

Paw pressure test: Mechanical hyperalgesia was measured as the threshold to a noxious mechanical stimulus. Nociceptive thresholds (NT) were determined in handled rats by a modification of the Randall-Selitto method (Kayser et al., 1990). Briefly, a constantly increasing pressure is applied to the rat hind paw until vocalization occurs. A Basile analgesimeter (Bioseb, France; stylus tip diameter, 1 mm) was used. A 600-g cut-off value was determined to prevent tissue damage.

Intrathecal Injection

Intrathecal injection was performed via manual lumbar puncture over one minute under anesthesia (isoflurane).

Drugs

Buprenorphine was purchased from Centravet. Scrambled control small interfering (siRNA) based on the Flt3 gene sequence and a pool of 4 specific siRNAs against Flt3 (F1t3-siRNA; ref # L-040111-00-0020) were obtained from Dharmacon. Buprenorphine (100 μg/kg) was administered twice daily subcutaneously for 4 consecutive days. Saline, an siRNA directed against FLT3 (2 μg/rat) or a scrambled siRNA (2 μg/rat) were administered once daily intrathecally for 4 consecutive days one hour before each opioid administration performed on the morning.

Statistical Analysis

Data are presented as mean±S.E.M. To evaluate the time-course effects of treatments and individual group comparisons, one-way and two-way ANOVA with repeated measurements were performed followed by Dunnett's post hoc test. Bonferroni's test was used for multiple comparisons between groups. A difference was accepted as significant if the probability that it occurred by chance alone was less than 5% (p<0.05).

Results

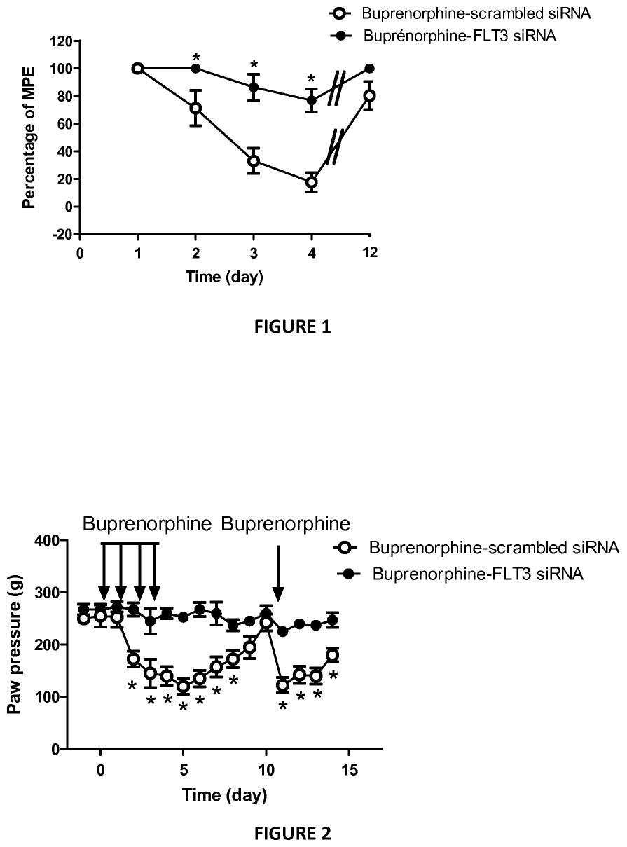

Repeated administrations of buprenorphine induced a progressive decrease in opioid-induced analgesia as showed by the decreased percentage of MPE from Do to D4 in control, scrambled siRNA-treated animals (FIG. 1). Intrathecal pre-treatment with the siRNA directed against FLT3, significantly reduced the decrease in buprenorphine analgesia.

### Example 2: Reduction by an Inhibitor of FLT3 Expression, of Mechanical Pain Hypersensitivity and Latent Pain Sensitization After Chronic Buprenorphine Treatment Materials & Methods

Animals

Male Sprague-Dawley rats (Janvier, Le Genest St Isle, France) weighing 175-199 g were used for the different experiments described here. Rats were kept under controlled environmental conditions (22° C., 60% relative humidity, 12 h light/dark cycle with lights on at 7:00 A.M., food and water ad libitum). To limit the stress induced by the experimental procedure, the general plan for an experimental phase was as follows:
arrival and housing of the animals: 4 days;
acclimation of the animals for 10 days to the experimental conditions in order to avoid any possibility of measurement bias being induced by stress;
evaluation of nociceptive threshold baseline
repeated opioid administration and evaluation of treatment effects

Paw Pressure Test

Mechanical hyperalgesia was measured as the threshold to a noxious mechanical stimulus. Nociceptive thresholds (NT) were determined in handled rats by a modification of the Randall-Selitto method (Kayser et al., 1990). Briefly, a constantly increasing pressure is applied to the rat hind paw until vocalization occurs. A Basile analgesimeter (Bioseb, France; stylus tip diameter, 1 mm) was used. A 600-g cut-off value was determined to prevent tissue damage.

Intrathecal Injection

Intrathecal injection was performed via manual lumbar puncture over one minute under anesthesia (isoflurane).

Drugs

Buprenorphine was purchased from Centravet. Scrambled control small interfering (siRNA) based on the Flt3 sequence and a pool of 4 specific siRNAs against Flt3 (F1t3-siRNA; ref # L-040111-00-0020) were obtained from Dharmacon. Buprenorphine (100 μg/kg) was administered twice daily subcutaneously for 4 consecutive days. An siRNA directed against FLT3 (2 μg/rat) or a scrambled siRNA (2 μg/rat) was administered once daily intrathecally for 4 consecutive days one hour before each buprenorphine administration performed on the morning. A single subcutaneous injection of buprenorphine (100 μg/kg) was performed when the nociceptive threshold returned to baseline values on D12.

Statistical Analysis

Data are presented as mean±S.E.M. To evaluate the time-course effects of treatments and individual group comparisons, one-way and two-way ANOVA with repeated measurements were performed followed by Dunnett's post hoc test. Bonferroni's test was used for multiple comparisons between groups. A difference was accepted as significant if the probability that it occurred by chance alone was less than 5% (P<0.05).

Results

A significant decrease in the nociceptive threshold, i.e. pain hypersensitivity, was observed on D2 in buprenorphine-treated animals and returned to baseline values on D9 in control, scrambled RNA-treated animals (FIG. 2). The administration of a single dose of buprenorphine precipitated the reappearance of sensory hypersensitivity, due to latent pain sensitization, for 2 days. The administration of the inhibitor of FLT3 expression (Flt3-targeted siRNA) completely prevented the development of buprenorphine-induced pain hypersensitivity and buprenorphine-revealed latent pain sensitization.

### Example 3: Flt3 Gene Deletion in the Mouse Abolishes Mechanical Pain Hypersensitivity Appearing After Chronic Administration of Morphine

Materials & Methods

Animals

Experiments were performed in male mice carrying a homozygous deletion of Flt3 (F1t3KO mice)²⁴ and their littermates (WT) weighing 25-30 g. All the procedures were approved by the French Ministry of Research (authorization #1006). Animals were maintained in a climate-controlled room on a 12 h light/dark cycle and allowed access to food and water ad libitum. To limit the stress induced by the experimental procedure, the general plan for an experimental phase was as follows:
arrival and housing of the animals: 4 days;
evaluation of nociceptive threshold baseline;
repeated opioid administration and evaluation of treatment effects

Nociceptive Testing

Tactile withdrawal threshold was determined in response to probing of the hindpaw with eight calibrated von Frey filaments (Stoeling, Wood Dale, Ill., USA) in logarithmically spaced increments ranging from 0.41 to 15 g (4-150 mN). Filaments were applied perpendicularly to the plantar surface of the paw. The 50% paw withdrawal threshold was determined in grams by the Dixon nonparametric test. The protocol was repeated until three changes in behavior occurred.

Drugs

Morphine was purchased from Francopia. Morphine (10 mg/kg) was administered subcutaneously.

Statistical Analysis

Data are presented as mean±S.E.M. To evaluate the time-course effects of treatments and individual group comparisons, one-way and two-way ANOVA with repeated measurements were performed followed by Dunnett's post hoc test. Bonferroni's test was used for multiple comparisons between groups. A difference was accepted as significant if the probability that it occurred by chance alone was less than 5% (P<0.05).

Results

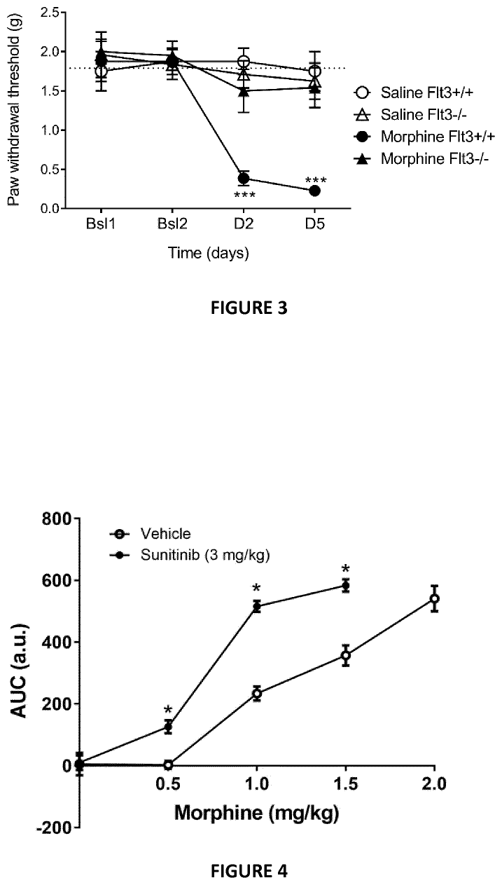

As shown in FIG. 3, in wild-type animals (F1t3+/+) mice, repeated morphine administrations induced a decrease in the nociceptive threshold, i.e. mechanical pain hypersensitivity, compared to animals treated with saline. In Flt3-deficient (F1t3−/−) animals, the nociceptive threshold remained unchanged, indicating that mechanical hypersensitivity has not developed in these animals.

### Example 4: Sunitinib, an RTKi or BDT001, an Inhibitor of the Interaction Between FLT3 and FL, Potentiates Morphine-Induced Analgesia and Permits Morphine-Dose Sparing

Materials & Methods

Animals

Male Sprague-Dawley rats (Janvier, Le Genest St Isle, France) weighing 175-199 g were used for the different experiments described here. Rats were kept under controlled environmental conditions (221° C., 60% relative humidity, 12 h light/dark cycle with lights on at 7:00 A.M., food and water ad libitum). To limit the stress induced by the experimental procedure, the general plan for an experimental phase was as follows:
arrival and housing of the animals: 4 days;
acclimatization of the animals for 10 days to the experimental conditions in order to avoid any possibility of measurement bias being induced by stress;
evaluation of nociceptive threshold baseline
morphine administration and evaluation of treatment effects

Nociceptive Testing

Paw pressure test: Mechanical hyperalgesia was measured as the threshold to a noxious mechanical stimulus. Nociceptive thresholds (NT) were determined in handled rats by a modification of the Randall-Selitto method (Kayser et al., 1990). Briefly, a constantly increasing pressure is applied to the rat hind paw until vocalization occurs. A Basile analgesimeter (Bioseb, France; stylus tip diameter, 1 mm) was used. A 600-g cut-off value was determined to prevent tissue damage.

Drugs

Morphine was purchased from Francopia. Morphine (0.5, 1, 1.5 or 2 mg/kg) was administered subcutaneously. The FLT3 inhibitor sunitinib was purchased from Sigma-Aldrich. BDT001 par obtained from Dr. Didier Rognan, Laboratoire d'Innovation Thérapeutique, CNRS/Faculté de Pharmacie, 67400 Illkirch, France. Sunitinib (3 mg/kg i.p.) or BDT001 (1 mg/kg) was administered intraperitoneally 2 h before morphine administration.

Statistical Analysis

The analgesic effects of morphine were represented via the calculation of the area under the curve (AUC) determined by the trapezoidal method. To evaluate the group effects of treatments, two-way ANOVA with repeated measurements were performed followed by Dunnett's post-hoc test. Newman-Keul's test was used for multiple comparisons between groups. A difference was accepted as significant if the probability that it occurred by chance alone was less than 5% (P<0.05).

Results

Morphine administration induced analgesia in a dose-dependent manner as shown by the calculation of the area under the curve on FIG. 4. Sunitinib, at a dose of 3 mg/kg, potentiated the analgesic effect of morphine at a dose of 1 mg/kg, as shown by shift to the left of the dose-response curve of morphine. For instance, the amount of analgesic effect produced by morphine at a dose of 1 mg/kg was doubled when combined with sunitinib. Moreover, the amount of analgesic effect of morphine at 1 mg/kg, combined with sunitinib, was identical to the amount of analgesia produced by morphine alone at a dose of 2 mg/kg. Thus, sunitinib was able to potentiate the analgesic effect of morphine so that the dose of morphine could be reduced by 50% without any loss of analgesic effect.

BDT001 (1 mg/kg), did not produced analgesia by itself: 30 min after administration, the pressure exerted on the hindpaw to obtain withdrawal was 250±7.4 g and 252.5±9.0 g (mean±S.E.M. of values obtained in 6 animals), in animals treated with saline and BDT001, respectively. By comparison, the pressure was 502.5±20.0 g, 30 min after administration of morphine (1.5 mg/kg).

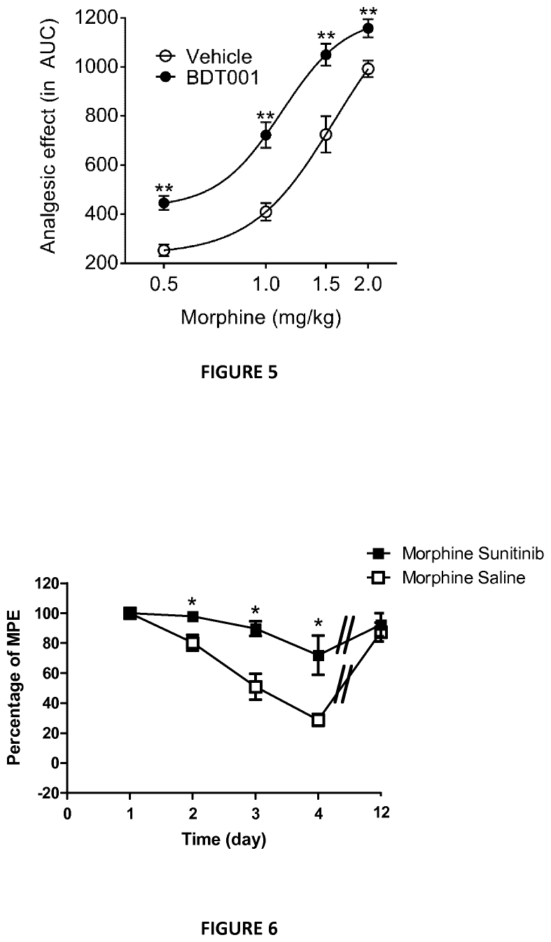

FIG. 5 shows the dose-response to morphine, expressed in AUC: the ED50 values (efficacious dose producing 50% of the maximal response) of morphine were 1.11 mg/kg and 1.56 mg/kg, without and with BDT001, respectively. The morphine-dose sparing effect of BDT001 was 30%.

### Example 5: Reduction by Sunitinib, Lestaurtinib, Two RTKi, or BDT001, an Inhibitor of the Interaction Between FLT3 and FL, of Tolerance After Chronic Morphine Treatment

The Materials & Methods were as in EXAMPLE 1, except that morphine (2 mg/kg) was administered twice daily subcutaneously for 4 consecutive days together with sunitinib (53 ng/rat) or lestaurtinib (175 ng/rat) injected intrathecally. In another experiment, BDT001 (1 mg/kg) was injected intraperitoneally together with morphine (2 mg/kg).

Results

Repeated administration of morphine (FIG. 6) induced a progressive decrease in morphine-induced analgesia as showed by the decreased percentage of MPE from Do to D4 in control animals. Intrathecal pre-treatment with the FLT3 inhibitor sunitinib significantly reduced the decrease in morphine analgesia. A similar effect was obtained with another FLT3 RTK inhibitor, lestaurtinib, injected intrathecally and BDT001, injected intraperitoneally.

### Example 6: Reduction by Sunitinib or BDT001 of Mechanical Pain Hypersensitivity and Latent Pain Sensitization After Chronic Morphine Treatment

Materials & Methods are as in EXAMPLE 2, except that morphine (2 mg/kg) was administered instead of buprenorphine and that sunitinib (3 mg/kg) was administered intraperitoneally. In another experiment, BDT001 (1 mg/kg) was injected intraperitoneally.

Results

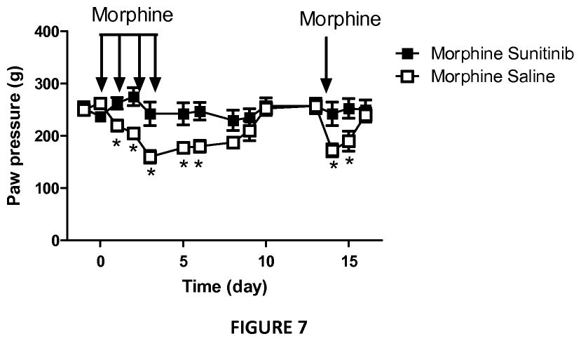

A significant decrease in the nociceptive threshold, i.e. pain hypersensitivity, was observed on D2 in morphine-treated animals and returned to baseline values on D9 in control, scrambled RNA-treated animals (FIG. 7). The administration of a single dose of morphine precipitated the reappearance of sensory hypersensitivity, due to latent pain sensitization, for 2 days. The administration of sunitinib completely prevented the development of morphine-induced pain hypersensitivity and morphine-revealed latent pain sensitization. Similar results were obtained with BDT001.

### REFERENCES

Throughout this application, various references describe the state of the art to which this invention pertains. The disclosures of these references are hereby incorporated by reference into the present disclosure
Boudreau, D., Von Korff, M., Rutter, C. M., Saunders, K., Ray, G. T., Sullivan, M. D., Campbell, C. I., Merrill, J. O., Silverberg, M. J., Banta-Green, C., et al. (2009). Trends in long-term opioid therapy for chronic non-cancer pain. Pharmacoepidemiol. Drug Saf. 18, 1166-1175.
Choi, V. W., Samulski, R. J., and McCarty, D. M. (2005). Effects of adeno-associated virus DNA hairpin structure on recombination. J. Virol. 79, 6801-6807.
Hassanein, M., Almahayni, M. H., Ahmed, S. O., Gaballa, S., and El Fakih, R. (2016). FLT3 Inhibitors for Treating Acute Myeloid Leukemia. Clin. Lymphoma Myeloma Leuk. 16, 543-549.
Holt, L. J., Herring, C., Jespers, L. S., Woolven, B. P., and Tomlinson, I. M. (2003). Domain antibodies: proteins for therapy. Trends Biotechnol. 21, 484-490.
Kuehn, B. M. (2009). Efforts aim to curb opioid deaths, injuries. JAMA 301, 1213-1215.
Li, Y., Li, H., Wang, M.-N., Lu, D., Bassi, R., Wu, Y., Zhang, H., Balderes, P., Ludwig, D. L., Pytowski, B., et al. (2004). Suppression of leukemia expressing wild-type or ITD-mutant FLT3 receptor by a fully human anti-FLT3 neutralizing antibody. Blood 104, 1137-1144.
McManus, M. T., and Sharp, P. A. (2002). Gene silencing in mammals by small interfering RNAs. Nat. Rev. Genet. 3, 737-747.
Rivat, C., Laulin, J.-P., Corcuff, J.-B., Célèrier, E., Pain, L., and Simonnet, G. (2002). Fentanyl enhancement of carrageenan-induced long-lasting hyperalgesia in rats: prevention by the N-methyl-D-aspartate receptor antagonist ketamine. Anesthesiology 96, 381-391.
Rivat, C., Laboureyras, E., Laulin, J.-P., Le Roy, C., Richebé, P., and Simonnet, G. (2007). Non-nociceptive environmental stress induces hyperalgesia, not analgesia, in pain and opioid-experienced rats. Neuropsychopharmacol. Off. Publ. Am. Coll. Neuropsychopharmacol. 32, 2217-2228.
Rivat C, Sar C, Mechaly I, Leyris JP, Diouloufet L, Sonrier C, Philipson Y, Lucas O, Mallié S, Jouvenel A, Tassou A, Haton H, Venteo S, Pin J P, Trinquet E, Charrier-Savournin F, Mezghrani A, Joly W, Mion J, Schmitt M, Pattyn A, Marmigère F, Sokoloff P, Carroll P, Rognan D, Valmier J., Inhibition of neuronal FLT3 receptor tyrosine kinase alleviates peripehral neuropathic pain in mice. Nat Commun, 9, 1042 (2018).
Tuschl, T., Zamore, P. D., Lehmann, R., Bartel, D. P., and Sharp, P. A. (1999). Targeted mRNA degradation by double-stranded RNA in vitro. Genes Dev. 13, 3191-3197.
Ward, E. S., Güssow, D., Griffiths, A. D., Jones, P. T., and Winter, G. (1989). Binding activities of a repertoire of single immunoglobulin variable domains secreted from Escherichia coli. Nature 341, 544-546.
Wu, Z., Asokan, A., and Samulski, R .J. (2006). Adeno-associated virus serotypes: vector toolkit for human gene therapy. Mol. Ther. J. Am. Soc. Gene Ther. 14, 316-327.
Zetsche, B., Gootenberg, J. S., Abudayyeh, O. O., Slaymaker, I. M., Makarova, K. S., Essletzbichler, P., Volz, S. E., Joung, J., van der Oost, J., Regev, A., et al. (2015). Cpf1 is a single RNA-guided endonuclease of a class 2 CRISPR-Cas system. Cell 163, 759-771.

1-17. (cancelled)

18. A method for treating pain in a patient in need thereof, consisting of:
(i) administering to a patient in need thereof an effective amount of FLT3 inhibitor during a first phase of treatment; and
(ii) then administering to said patient in need thereof an effective amount of an opioid in a second phase of treatment, wherein the first and second phase of treatment may overlap or not and wherein the sequence (i) and (ii) may be repeated.

19. The method according to claim 18, wherein the patient has not previously been treated by an opioid.

20. The method according to claim 18, wherein the patient has previously been treated by an opioid, and an opioid-induced side-effect occurred.

21. The method according to claim 18, wherein the patient has previously been treated by an opioid, and an ineffectiveness or a decline in a prior opioid treatment effectiveness or an opioid tolerance occurred.

22. The method according to claim 20, wherein, the side effect is selected from opioid-induced hyperalgesia or opioid-induced latent pain sensitization.

23. he method according to claim 18, wherein said FLT3 inhibitor is a receptor tyrosine kinase inhibitor (RTK1).

24. The method according to claim 23, wherein the RTKI is selected from the group consisting of lestaurtinib (CEP-701), sunitinib (SU-11248), midostaurin (PKC412), semaxinib (SU-5416), quizartinib (AC220), tandutinib (MLN518), sorafenib (BAY 43-9006), gilteritinib and crenolanib (CP-868).

25. The method according to claim 18, wherein said inhibitor is an inhibitor of the FL/FLT3 interaction.

26. The method according to claim 25, wherein the inhibitor of the FL/FLT3 interaction is a compound of formula (I) 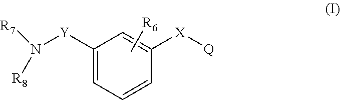
wherein:
X is CO—NH or triazolyl,
Y represents SO₂,
Q is selected from a group of formula: 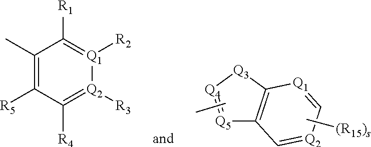
Q₁ and Q₂ are CH,
Q₃ is selected from O, S, N and NH,
Q₄ is selected from C and N, and CO,
Q₅ is selected from C and N,
R₆ is selected from H, OH, alkyl, hydroxyalkyl and alkoxy,
R₁ represents OH,
R₂ represents H,
R₃ is selected from H, OR₁₁, halo and O—(CH₂)ₚ—O-alkyl;
R₄ is selected from H, alkyl, halo, CN, trifluoromethyl, CO-alkyl, phenyl and benzyl;
with the proviso that one from R₃ and R₄ is H;
R₅ is H, or
two from R₂ and R₃ or R₃ and R₄ or R₄ and R₅ together with the carbon atoms to which they are attached form an aromatic ring comprising 5 to 6 members, and the others from
R₂ to R₅ represent H,
R₇ and R₈ represent alkyl, or
R₇ and R₈ together with the N atom to which they are attached form a group of formulae: 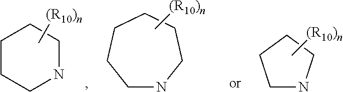
wherein R₁₀ is selected from H, alkyl, halo, trifluoromethyl, aryl and hydroxyalkyl or two adjacent R₁₀ groups together with the cyclic atoms to which they are attached form an aryl group; or
R₇ and R₈ together with the N atom to which they are attached form a group of formula: 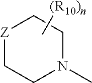
wherein Z is a NR₁₄ group, wherein R₁₄ is selected from phenyl, benzyl and pyrimidyl, or
R₇ is H and R₈ is cycloalkyl, preferably cyclohexyl and adamantyl,
R₁₁ is H or alkyl,
R₁₅ represents a group selected from H, halo, OH and alkoxy,
s is 0, 1, 2 or 3, and
n is 1.

27. The method according to claim 26, wherein said inhibitor is N-(5-chloro-2-hydroxyphenyl)-3-(piperidin-1-ylsulfonyl)benzamide (BDT001).

28. The method according to claim 18, wherein said FLT3 inhibitor is an inhibitor of FLT3 gene expression.

29. The method according to claim 18, wherein said FLT3 inhibitor is an anti-FL antibody or an anti-FLT3 antibody.

30. The method according to claim 18, wherein the opioid is selected from the group consisting of fentanyl, alfentanil, codeine, pethidine, remifentanyl, morphine, tramadol, buprenorphine, nalbuphine, morphine sulphate, hydromorphone hydrochloride and coated morphine sulphate.

31. A pharmaceutical combination comprising an FLT3 inhibitor and an opioid.

32. The pharmaceutical combination according to claim 31, wherein the FLT3 inhibitor is selected from a receptor tyrosine kinase inhibitor (RTKI), an inhibitor of the FL/FLT3 interaction, an inhibitor of FLT3 gene expression or an anti-FL antibody or an anti-FLT3 antibody.

33. The pharmaceutical combination according to claim 31, wherein the opioid is selected from the group consisting of fentanyl, alfentanil, codeine, pethidine, remifentanyl, morphine, tramadol, buprenorphine, nalbuphine, morphine sulphate, hydromorphone hydrochloride and coated morphine sulphate.

34. A method for treating pain in a patient in need thereof, consisting of administering to said patient a pharmaceutical combination comprising an FLT3 inhibitor and an opioid in a separate administration, an administration spread out over time or a simultaneous administration to said patient.

35. The method according to claim 34, wherein the FLT3 inhibitor is a compound of formula (I) 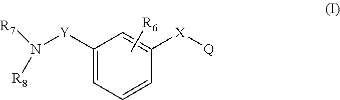
wherein:
X is CO—NH or triazolyl,
Y represents SO₂,
Q is selected from a group of formula: 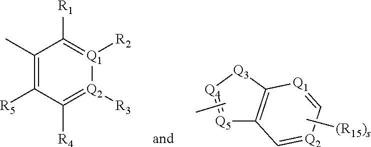
Q₁ and Q₂ are CH,
Q₃ is selected from O, S, N and NH,
Q₄ is selected from C and N, and CO,
Q₅ is selected from C and N,
R₆ is selected from H, OH, alkyl, hydroxyalkyl and alkoxy,
R₁ represents OH,
R₂ represents H,
R₃ is selected from H, OR₁₁, halo and O—(CH₂)ₚ—O-alkyl;
R₄ is selected from H, alkyl, halo, CN, trifluoromethyl, CO-alkyl, phenyl and benzyl;
with the proviso that one from R₃ and R₄ is H;
R₅ is H, or
two from R₂ and R₃ or R₃ and R₄ or R₄ and R₅ together with the carbon atoms to which they are attached form an aromatic ring comprising 5 to 6 members, and the others from
R₂ to R₅ represent H,
R₇ and R₈ represent alkyl, or
R₇ and R₈ together with the N atom to which they are attached form a group of formulae: 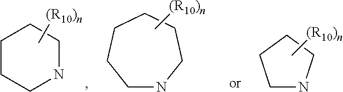
wherein R₁₀ is selected from H, alkyl, halo, trifluoromethyl, aryl and hydroxyalkyl or two adjacent R₁₀ groups together with the cyclic atoms to which they are attached form an aryl group; or
R₇ and R₈ together with the N atom to which they are attached form a group of formula: 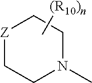
wherein Z is a NR₁₄ group, wherein R₁₄ is selected from phenyl, benzyl and pyrimidyl, or
R₇ is H and R₈ is cycloalkyl, preferably cyclohexyl and adamantyl,
R₁₁ is H or alkyl,
R₁₅ represents a group selected from H, halo, OH and alkoxy;
s is 0, 1, 2 or 3, and
n is 1.

36. The method according to claim 34, wherein the FLT3 inhibitor is N-(5-chloro-2-hydroxyphenyl)-3-(piperidin-1-ylsulfonyl)benzamide.

37. A pharmaceutical kit intended for treating pain, comprising:
a first galenical formulation comprising an FLT3 inhibitor, and
a second galenical formulation comprising an opioid.

38. The pharmaceutical kit according to claim 37, wherein wherein the FLT3 inhibitor is a compound of formula (I) 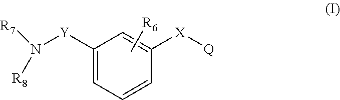
wherein:
X is CO—NH or triazolyl,
Y represents SO₂,
Q is selected from a group of formula: 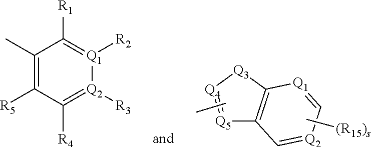
Q₁ and Q₂ are CH,
Q₃ is selected from O, S, N and NH,
Q₄ is selected from C and N, and CO,
Q₅ is selected from C and N,
R₆ is selected from H, OH, alkyl, hydroxyalkyl and alkoxy,
R₁ represents OH,
R₂ represents H,
R₃ is selected from H, OR₁₁, halo and O—(CH₂)ₚ—O-alkyl,
R₄ is selected from H, alkyl, halo, CN, trifluoromethyl, CO-alkyl, phenyl and benzyl;
with the proviso that one from R₃ and R₄ is H;
R₅ is H, or
two from R₂ and R₃ or R₃ and R₄ or R₄ and R₅ together with the carbon atoms to which they are attached form an aromatic ring comprising 5 to 6 members, and the others from
R₂ to R₅ represent H,
R₇ and R₈ represent alkyl, or
R₇ and R₈ together with the N atom to which they are attached form a group of formulae: 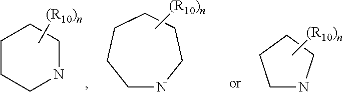
wherein R₁₀ is selected from H, alkyl, halo, trifluoromethyl, aryl and hydroxyalkyl or two adjacent R₁₀ groups together with the cyclic atoms to which they are attached form an aryl group; or
R₇ and R₈ together with the N atom to which they are attached form a group of formula: 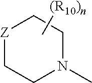
wherein Z is a NR₁₄ group, wherein R₁₄ is selected from phenyl, benzyl and pyrimidyl, or
R₇ is H and R₈ is cycloalkyl, preferably cyclohexyl and adamantyl,
R₁₁ is H or alkyl,
R₁₅ represents a group selected from H, halo, OH and alkoxy,
s is 0, 1, 2 or 3, and
n is 1.

39. The pharmaceutical kit according to claim 37, wherein wherein the FLT3 inhibitor is N-(5-chloro-2-hydroxyphenyl)-3-(piperidin-1-ylsulfonyl)benzamide.
